(19) United States
(12) Patent Application Publication
(10) Pub. No.: US 2020/0138563 A1
(43) Pub. Date: May 7, 2020

(54) RETINA REGENERATION WITH A TISSUE-AND-TECHNOLOGY PROSTHESIS

(71) Applicant: THE JOHNS HOPKINS UNIVERSITY, Baltimore, MD (US)

(72) Inventors: Yassine Daoud, Clarksville, MD (US); Maria Canto-Soler, Washington, DC (US); Ralph Etienne-Cummings, Washington, DC (US); Mandeep Singh, Baltimore, MD (US); Gerard Lutty, Hyattsville, MD (US)

(21) Appl. No.: 16/477,594

(22) PCT Filed: Jan. 16, 2018
(86) PCT No.: PCT/US2018/013771

§ 371 Date: Jul. 12, 2019

Related U.S. Application Data
(60) Provisional application No. 62/445,911, filed on Jan. 13, 2017.

(51) Int. Cl. A61F 2/14; A61N 1/32; A61N 1/36

(52) U.S. Cl. CPC A61F 2/14; A61F 2250/0001; A61N 1/36046; A61N 1/326

(57) ABSTRACT

An embodiment in accordance with the present invention includes 3D retinal tissue generated in a laboratory. The 3D retinal tissue is coupled to an engineered microelectronic chip. The 3D retinal tissue together with the engineered microelectronic chip enable retinal regeneration and vision restoration for patients with retinal cell damage. The engineered microelectronic chip sends electrical signals to specific parts of the 3D retinal tissue for stimulating and recording both the 3D retinal tissue and the cells in the patient's own retina. The chip may be absorbable or removable once connection is made between the 3D retinal tissue and the patient's own remaining retinal tissue.

### CROSS REFERENCE TO RELATED APPLICATIONS

This application claims the benefit of U.S. Provisional Patent Application No. 62/445,911, filed Jan. 13, 2017, which is incorporated by reference herein, in its entirety.

### FIELD OF THE INVENTION

The present invention relates generally to medical devices. More particularly, the present invention relates to a device and method for retina regeneration with a tissue-and-technology prosthesis.

### BACKGROUND OF THE INVENTION

Retinal degenerative diseases are a group of clinical conditions in which the damage, dysfunction and death of retinal photoreceptor cells lead to vision loss, and sometimes, total blindness. The development of human induced pluripotent stem cells (hiPSCs) roused great hope for their potential use in therapeutic treatments. Virtually all preclinical studies aimed at replacing photoreceptors have transplanted a suspension of dissociated photoreceptor cells. In the host retina, however, the transplanted cells have failed to survive in sufficient numbers or to become functionally integrated. A new approach is needed.

Many forms of blindness result from the dysfunction or loss of retinal photoreceptors. Stem cells, especially induced pluripotent stem cells (iPSCs), may hold great promise for the modeling and/or therapy of diseases. Previous work has shown that, when provided with the appropriate cues, mouse and human pluripotent stem (ES) cells in culture can develop into a 3-dimensional eyecup that remarkably resembles the vertebrate eye, including differentiation of photoreceptor-like cells. The current limitation here is that, even though human pluripotent derived 3D retinal tissue with functional photoreceptors is feasible, there is still the need for developing a “transplantable” version of the 3D retina tissue and the ability to induce functional integration of the transplanted 3D retinal tissue into the host retina.

Accordingly, there is a need in the art for a tissue-and-technology prosthesis for retinal regeneration.

### SUMMARY OF THE INVENTION

The foregoing needs are met, to a great extent, by the present invention which provides a retinal prosthesis. The retinal prosthesis includes in vitro retinal tissue. The retinal prosthesis also includes a microchip configured to provide a scaffold for the in vitro retinal tissue. The microchip is configured to provide stimulation of the in vitro retinal tissue and record electrical signals from the in vitro retinal tissue. The microchip is configured to be coupled to a patient's residual retinal tissue.

In accordance with an aspect of the present invention, the retinal prosthesis further includes a computing device configured for control of the microchip. The microchip is configured to communicate with the computing device. The communication is achieved using one selected from a group including wireless or wired communication. The retinal tissue includes one selected from a group consisting of RPE/NR tissue and NR tissue. The lab-generated tissue is embedded in one selected from a group of a hydrogel and PEDOT polymer. The lab-generated tissue is formed from human induced pluripotent stem cells. The microchip includes photosensitive elements. The retinal prosthesis includes a control and visualization connection. The microchip is formed from a biodegradable material.

In accordance with another aspect of the present invention, a method of retinal regeneration includes growing iPS-derived retinal pigment epithelium (RPE) and photoreceptor cells on a porous biological scaffold. The porous biological scaffold contains instrumentation. The method includes guiding growth of the cells with electrical stimulation. The method also includes validating functionality of the RPE and photoreceptor cells as they grow.

In accordance with yet another aspect of the present invention, the method includes transplanting the RPE and photoreceptor cells and biological scaffold to a host retina. The method further includes stimulation of the RPE and photoreceptor cells to encourage formation of connections between the RPE and photoreceptor cells and the host retina. The method further includes monitoring functionality of the transplanted cells. The method includes using recoding electronics for monitoring. The method includes forming the biological scaffold from a bioerodible material. The method also includes using a transparent encapsulation material for the biological scaffold. The method includes receiving communication from the biological scaffold. The method includes using a microchip to provide communication to and from the biological scaffold. Further, the method includes using the microchip to stimulate cell growth.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic diagram of a neural-retinal instrumented scaffold hybrid, according to an embodiment of the present invention.

FIG. 2 illustrates a schematic diagram of an RPE neural-retinal instrumented scaffold hybrid, according to an embodiment of the present invention.

FIG. 3 illustrates a schematic diagram of an implanted scaffold containing lab-grown retinal tissue, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims. U.S. Patent Application Publication Number 2016/0333312 is hereby incorporated by reference.

An embodiment in accordance with the present invention includes 3D retinal tissue generated in a laboratory. The 3D retinal tissue is coupled to an engineered microelectronic chip. The 3D retinal tissue together with the engineered microelectronic chip enable retinal regeneration and vision restoration for patients with retinal cell damage. The engineered microelectronic chip sends electrical signals to specific parts of the 3D retinal tissue for stimulating and recording both the 3D retinal tissue and the cells in the patient's own retina. The chip may be absorbable or removable once connection is made between the 3D retinal tissue and the patient's own remaining retinal tissue.

3D retinal tissue can be generated ex vivo from inducible pluripotent stem cells (iPS cells). iPS cells are derived from a small, autologous, skin or blood biopsy and then coaxed, in the lab, to differentiate into mature light-sensitive photoreceptors. Generating photoreceptor tissue in the lab, rather than merely a suspension of photoreceptor cells, provides an already-integrated biological unit that will be suitable for transplantation. Visual outcomes are vastly improved by implanting tissue rather than cells, and are further enhanced by supporting the integration and function of the transplanted tissue with a microelectronic chip. The present invention provides interface chips that both stimulate, and record from, neurons in the cortex and the peripheral nervous system. The stimulation and recording systems of the present invention are developed to occupy a few tens of micrometers in diameter. The present invention also includes a combination of microelectronic chips developed specifically for the retina, with neural interface electronics, allow for the production of a visual prostheses with a large number of photodetectors and stimulation and recording electrodes. The combination of lab-grown retinal tissue with microelectronics of the present invention yields a leap for functional visual prostheses.

The present invention includes a “photoreceptor/retinal pigment epithelium (RPE) tissue construct”—a scaffold embedded with functional cells—that is suitable for transplantation. This tissue is implanted in conjunction with a microelectronic chip specifically for the purpose of supporting the tissue's survival and functional integration.

One embodiment of the present invention includes growing, in the lab, iPS-derived RPE and photoreceptor cells on a porous biological scaffold which contains instrumentation (i.e., tooled stimulation and recording electronics). Electrical stimulation would guide the growth of the cells, while neural recording electronics would validate the functionality of the RPE/photoreceptor tissue as it grows. When grown to the optimal differentiation stage and demonstrated to be functional, the entire scaffold would be transplanted sub-retinally into the macula. After transplantation, stimulation is used, as well as appropriately engineered stem cells, to encourage the formation of connections between the photoreceptors in the scaffold and the host retina. Recording electronics allow for monitoring functionality on an ongoing basis. In some embodiments, the scaffold may be constructed from bioerodible material that dissolves over time, leaving only the microelectronics and control wiring in the regenerated biological tissue. These remnants do not affect vision as, by that time, the regenerated photoreceptors have connected with the host retinal cells in a way that restores visual function.

For fabricating the instrumented scaffold, developing an encapsulation material that is transparent, allows the electrodes to penetrate through it and can survive for many years or decades. The present invention enables the transplanted outer retinal tissue (photoreceptor/RPE layer) to overcome the gliotic seal barrier and achieve functional integration with the inner retina. The present invention enables the patient with the sensation of depth, a critical aspect of vision. The present invention also refines the signal perceived by the retinal cells for sharper image quality. Additionally, the present invention, incorporates image and surrounding pattern recognition software. Another aspect of the present invention ensures that the retinal prosthesis, like a real retina, can learn from and adapt to the individual's environment. The microchip functions, in some embodiments, can be expanded beyond stimulation and recording, such as excitation/inhibition circuits.

Generated from human iPS cells the first “3D human retina in a dish” of the present invention, unlike other prototypes, have functional photoreceptors. An efficient process for generating RPE tissue from human iPS cells has been developed. It is also possible to induce painless retinal scarring in animal models, which allows for testing of restored visual function in implanted animals; and, successfully transplanted photoreceptor cells into animal models using the subretinal injection technique.

As discussed above, the present invention combines biological/electrostimulation strategy for regenerating the retina. This strategy will join RPE-photoreceptor matrix structures that are grown in culture, with a chip technology that can promote re-establishment of functional inner retinal circuitry and stimulate ganglion cells. The present invention provides synthesized neural networks at multiple physical scales in hybrid animate/inanimate technologies—networks that can transduce, adapt, compute, learn, and function in a closed loop.

The present invention includes individual tissue patches of 1 sq. mm to 9 sq. mm of retinal pigment epithelium (RPE)/Neural-Retina (NR)/Instrumented-Scaffold hybrid structure that are implantable into the patient's eye. The preferred embodiment is a tissue patch of approximately 4 sq. mm. The thickness of the RPE/NR/Scaffold hybrid construct is in the range of approximately 30-100 microns. The instrumented scaffold includes of a Poly(3,4-ethylenedioxythiophene) (PEDOT) (a custom-designed polymer) matrix with conductive channels that allow electrical signals to be sent to specific parts of the matrix for stimulating and recording, extracellularly, the RPE/Neural-Retina (RPE/NR) and the cells in the patient's own retina. Based on its composition, the PEDOT matrix that forms the scaffold may be dissolvable or bioerodible over time.

The electrically conductive channels on the scaffold are connected, via gold (or other biologically suitable) wire bonds (on the scale of approximately 5 microns in diameter), to a silicon chip. Specifically, in a preferred embodiment, the present invention will include a Silicon-On-Insulator chip, with the carrier substrate etched away. This structure facilitates hermetical sealing. The chip contains integrated electronics to: provide multi-phasic stimulation currents to extracellularly stimulate the implanted RPE/NR or the residual retinal cells in the patient; to detect and amplify the neural activity from the RPE/NR or the residual retinal cells in the patient; wirelessly communicate power and data (for controlling the chip for stimulation, and to visualize the recorded neural data); support circuitry, such as bias generators, glue logic, references, etc. . . . to make the chip completely self-contained for implantable, bi-directional neural stimulation and recording. The chip size is on the scale of approximately 2-9 sq. mm.

If the PEDOT matrix is configured to dissolve, after the time, the surgeon will remove the electronic chip from the eye. If the PEDOT matrix is NOT configured to dissolve, the surgeon may chose to leave the silicon chip in the eye (provided that it is appropriately placed to not block vision and cause damage), or may also remove the chip, after connection between the RPE/NR and the residual retina has been made and experimentation has proven the function of the implant.

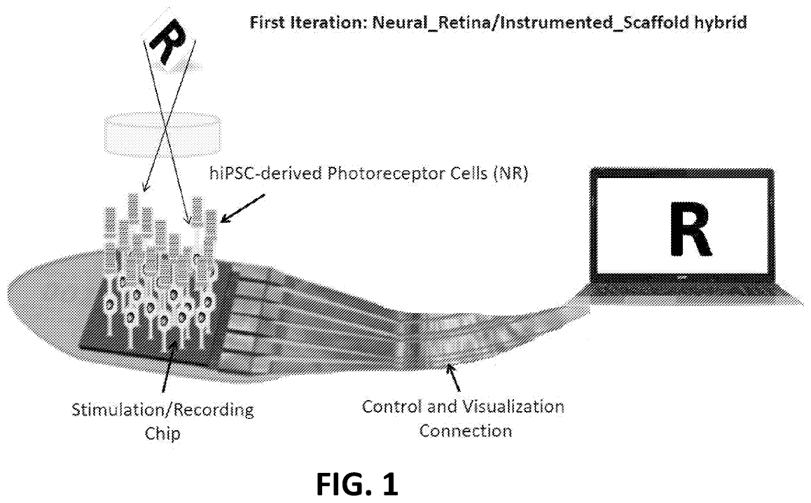

FIG. 1 illustrates a schematic diagram of a neural-retinal instrumented scaffold hybrid, according to an embodiment of the present invention. As illustrated in FIG. 1, the present invention includes patches of neural-retina tissue and an instrumented-scaffold hybrid structure that are implantable into an eye of the patient. In some embodiments, the tissue component of the hybrid is formed from human induced pluripotent stem cells (hiPSC)-derived neural-retinal tissue containing functional photoreceptor cells embedded in either hydrogel, PEDOT polymer, or a combination of both hydrogel and PEDOT polymer. While hydrogel and PEDOT are included herein as exemplary substrates for the NR tissue, any substrate known to or conceivable to one of skill in the art could also be used. It is also possible that the tissue is coupled to the microchip without the use of an additional substrate, or the microchip surface is formed from a substrate conducive to the maintenance of the NR tissue. The instrumented scaffold includes a PEDOT polymer matrix with conductive channels that will allow electrical signals to be sent to specific parts of the matrix for stimulation and recording. While PEDOT is disclosed herein as the matrix for forming the instrumented scaffold, any suitable material known to or conceivable to one of skill in the art can be used. The instrumented scaffold is also a light-sensitive microchip that contains photosensitive elements.

As illustrated in FIG. 1, the instrumented scaffold is coupled to the stimulation and recording chip and the stimulation and recording chip is coupled to a computing device via a control and visualization connection. The control and visualization connection can be wired, wireless, or a hybrid of the two. An internet, intranet, or other suitable network connection can be used for communication between the stimulating and recording chip and the computing device. The computing device can take the form of any suitable computing device known to or conceivable to one of skill in the art. The computing device is programmed to execute stimulation and recording of signals from the NR tissue or loaded with a non-transitory computer readable medium configured to execute the stimulation and recording associated with the present invention. FIG. 1 further shows the use of electronic stimulation to promote functional integration of transplanted hiPSC-derived retinal tissue and electronic neural recording to monitor the tissue.

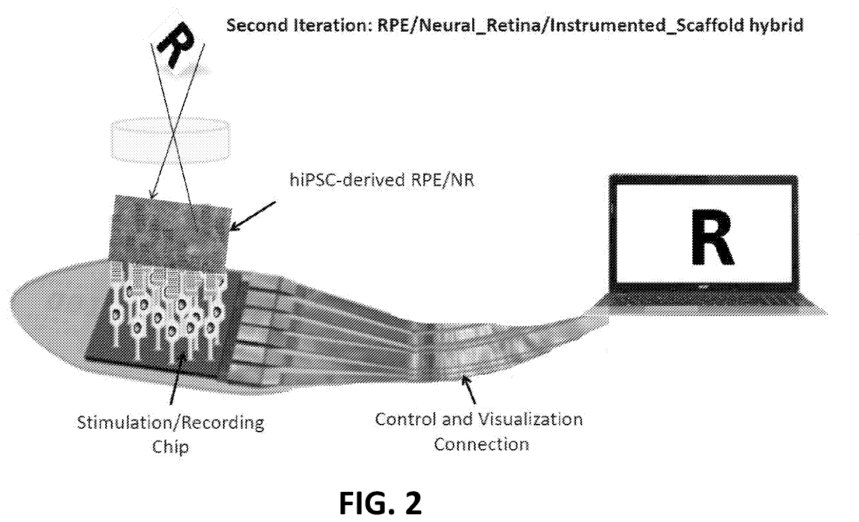

FIG. 2 illustrates a schematic diagram of an RPE neural-retinal instrumented scaffold hybrid, according to an embodiment of the present invention. As illustrated in FIG. 2, the present invention includes patches of RPE and NR tissue and an instrumented-scaffold hybrid structure that are implantable into an eye of the patient. In the embodiment of the present invention illustrated in FIG. 2, the tissue component of the hybrid is composed of hiPSC-derived RPE and NR containing functional photoreceptor cells embedded in either hydrogel, PEDOT polymer, or a combination of both. While hydrogel and PEDOT are included herein as exemplary substrates for the RPE and NR tissue, any substrate known to or conceivable to one of skill in the art could also be used. It is also possible that the tissue is coupled to the microchip without the use of an additional substrate, or the microchip surface is formed from a substrate conducive to the maintenance of the RPE and NR tissue. The instrumented scaffold includes a PEDOT polymer matrix with conductive channels that will allow electrical signals to be sent to specific parts of the matrix for stimulation and recording. While PEDOT is disclosed herein as the matrix for forming the instrumented scaffold, any suitable material known to or conceivable to one of skill in the art can be used. The instrumented scaffold is also a light-sensitive microchip that contains photosensitive elements.

Further, as illustrated in FIG. 2, the instrumented scaffold is coupled to the stimulation and recording chip and the stimulation and recording chip is coupled to a computing device via a control and visualization connection. The control and visualization connection can be wired, wireless, or a hybrid of the two. An internet, intranet, or other suitable network connection can be used for communication between the stimulating and recording chip and the computing device. The computing device can take the form of any suitable computing device known to or conceivable to one of skill in the art. The computing device is programmed to execute stimulation and recording of signals from the RPE and NR tissue or loaded with a non-transitory computer readable medium configured to execute the stimulation and recording associated with the present invention. FIG. 2 further shows the use of electronic stimulation to promote functional integration of transplanted hiPSC-derived retinal tissue and electronic neural recording to monitor the tissue. The tissue component of the hybrid takes the form of a patch of either RPE/NR tissue construct or just NR tissue embedded in hydrogel or a PEDOT polymer, or a combination of both.

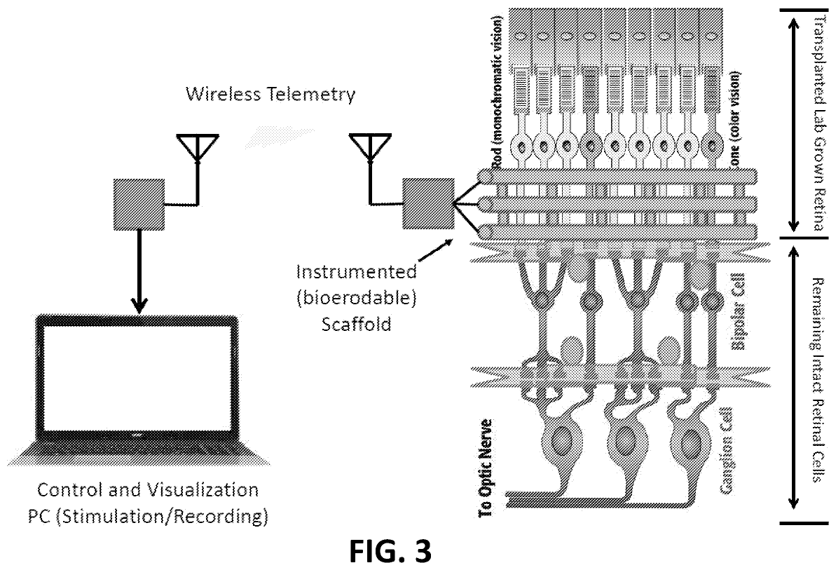

FIG. 3 illustrates a schematic diagram of an implanted scaffold containing lab-grown retinal tissue, according to an embodiment of the present invention. As illustrated in FIG. 3, the present invention includes a layered, implantable hybrid chip that is configured to communicate with a control and visualization personal computer or other computing device. FIG. 3 illustrates an embodiment configured with wireless communication. However, any type of wired, wireless, or hybrid communication known to or conceivable by one of skill in the art can also be used, including Bluetooth™, RFID, or other communication modality. FIG. 3 also illustrates the layered structure of the transplanted, lab-grown retinal tissue, the instrumented scaffold or microchip, and the remaining, intact retinal cells of the patient's own eye. The layer of transplanted, lab-grown retinal tissue, includes rod (monochromatic vision) cells and cone (color vision) cells. This tissue is coupled to a first side of the instrumented scaffold. The instrumented scaffold can be configured to be bioerodible, removable, or any other configuration known to or conceivable to one of skill in the art. The second side of the instrumented scaffold is coupled to bipolar cells and in turn ganglion cells that are in communication with the optic nerve.

The steps and analysis of the present invention can be carried out using a computing device, smartphone, a tablet, internet or cellular enabled device, computer, or non-transitory computer readable medium. Indeed, any suitable method of control and execution known to or conceivable by one of skill in the art could be used. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computing device designed specifically for this purpose. The computing device can be unique to the present invention and designed specifically to carry out the method of the present invention.

The many features and advantages of the invention are apparent from the detailed specification, and thus, it is intended by the appended claims to cover all such features and advantages of the invention, which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A retinal prosthesis comprising:
in vitro retinal tissue; and
a microchip configured to provide a scaffold for the in vitro retinal tissue, wherein the microchip is configured to provide stimulation of the in vitro retinal tissue and is configured record electrical signals from the in vitro retinal tissue, wherein the microchip is configured to be coupled to a patient's residual retinal tissue.

2. The retinal prosthesis of claim 1 further comprising a computing device configured for control of the microchip.

3. The retinal prosthesis of claim 2 further comprising the microchip being configured to communicate with the computing device.

4. The retinal prosthesis of claim 3 wherein the communication between the microchip and the computing device is achieved using one selected from a group consisting of wireless or wired communication.

5. The retinal prosthesis of claim 1, wherein the in vitro retinal tissue comprises one selected from a group consisting of RP retinal pigment epithelium (RPE)/Neural-Retina (NR) tissue and NR tissue.

6. The retinal prosthesis of claim 1 further comprising the in vitro retinal tissue being embedded in one selected from a group consisting of a hydrogel and PEDOT polymer.

7. The retinal prosthesis of claim 1 wherein the in vitro retinal tissue comprises human induced pluripotent stem cells.

8. The retinal prosthesis of claim 1 wherein the microchip comprises photosensitive elements.

9. The retinal prosthesis of claim 1 further comprising a control and visualization connection.

10. The retinal prosthesis of claim 1 wherein the microchip is formed from a biodegradable material.

11. A method of retinal regeneration comprising:
growing iPS-derived retinal pigment epithelium (RPE) and photoreceptor cells on a porous biological scaffold, wherein the porous biological scaffold contains instrumentation;
guiding growth of the RPE and photoreceptor cells with electrical stimulation; and
validating functionality of the RPE and photoreceptor cells as they grow.

12. The method of claim 11 further comprising transplanting the RPE and photoreceptor cells and porous biological scaffold to a host retina.

13. The method of claim 12 further comprising stimulation of the RPE and photoreceptor cells to encourage formation of connections between the RPE and photoreceptor cells and the host retina.

14. The method of claim 13 further comprising monitoring functionality of the transplanted cells.

15. The method of claim 14 further comprising using recoding electronics for monitoring.

16. The method of claim 11 further comprising forming the porous biological scaffold from a bioerodible material.

17. The method of claim 11 further comprising using a transparent encapsulation material for the porous biological scaffold.

18. The method of claim 11 further comprising receiving communication from the porous biological scaffold.

19. The method of claim 18 further comprising using a microchip to provide communication to and from the porous biological scaffold.

20. The method of claim 19 further comprising using the microchip to stimulate cell growth.
